(19) United States
(12) Patent Application Publication
(10) Pub. No.: US 2018/0071354 A1
(43) Pub. Date: Mar. 15, 2018

(54) SELECTIVE BREEDING METHOD OF ANTITUMOR/ANTICANCER CAMELLIA SINENSIS STRAIN, AND TEA PRODUCT OF SUCH STRAIN

(71) Applicant: FUJIAN AGRICULTURE AND FORESTRY UNIVERSITY, Fuzhou (CN)

(72) Inventors: Jingui ZHENG, Fuzhou (CN); Jinke LIN, Fuzhou (CN); Jiangfan YANG, Fuzhou (CN); Tuansheng CHEN, Fuzhou (CN); Sufeng LIAO, Fuzhou (CN); Jianghong LIU, Fuzhou (CN)

(21) Appl. No.: 15/814,831

(22) Filed: Nov. 16, 2017

Related U.S. Application Data
(63) Continuation-in-part of application No. PCT/CN2016/084936, filed on Jun. 6, 2016.

(30) Foreign Application Priority Data
Jul. 15, 2015 (CN) 201510414307.0

(51) Int. Cl. A61K 36/82; A01H 1/04; A01H 5/12

(52) U.S. Cl. CPC A61K 36/82; A01H 5/12; A01H 1/04

(57) ABSTRACT

Disclosed herein are a selective breeding method of an antitumor/anticancer Camellia sinensis strain and a tea product produced from such strain. Also disclosed herein are a pharmaceutical composition and a supplement, each of which includes the tea product. The selective breeding method includes: conducting a hybridization process so as to obtain progenies of Camellia sinensis; subjecting the progenies of Camellia sinensis to determination of bioactive ingredient contents; and selecting, from the progenies of Camellia sinensis, an antitumor/anticancer Camellia sinensis strain which meets five standards of bioactive ingredients.

### CROSS-REFERENCE TO RELATED APPLICATION

This application is a bypass continuation-in-part application of International Application No. PCT/CN2016/084936 filed on Jun. 6, 2016, which claims priority of Chinese Patent Application No. 201510414307.0, filed on Jul. 15, 2015. The entire content of each of the international and Chinese patent applications is incorporated herein by reference.

### FIELD

The present disclosure relates to germplasm resources of Camellia sinensis, and more particularly to a selective breeding method of an antitumor/anticancer Camellia sinensis strain and a tea product of such strain.

### BACKGROUND

It is well-known that tea leaves have antioxidant, antitumor, anti-aging, anti-virus, antibacterial, antihypertensive, antihyperlipidemic, and antihyperglycemic effects in terms of pharmaceutical use or dietary supplementary use. Tea leaves may contain more than 400 organic compounds and more than 15 inorganic minerals, among which the major antitumor/anticancer ingredients are tea polyphenols and oxidized products thereof, caffeine, tea pigments, L-theanine.

The major components of tea polyphenols are catechins, which may be monomeric, oligomeric, or polymeric. Epicatechin gallate (ECG) and epigallocatechin gallate (EGCG), which are ester-type catechins, are present in a higher amount and together account for about 75% of the total catechin content, and have the strongest pharmacological activity. As reported by Chen, Jankun et al., and Tan et al., ester-type catechins are able to inhibit growth of cancer cells and to induce differentiation and apoptosis of cancer cells, thereby having a strong anticancer effect (see Chen (2003), Journal of Tea Science, 23(2):83-93; Jankun et al. (1997), Nature, 387:561; and Tan et al. (2000), Cancer Letters, 158:1-6).

Du et al. and Chen et al. have found that EGCG is the most powerful catechin monomer which can suppress mammary gland cancer, gastric cancer, pancreatic cancer, prostate cancer, colon cancer, and melanoma (see Du et al. (2012), Nutrients, 4:1679-1691; and Chen et al. (2008), Histol Histopathol., 23(4):487-496), and Lamber et al., Tachibana et al., and Shimizu et al. further stated that EGCG may prevent formation and metastasis of tumors through various pathways (see Lambert et al. (2005), Am. J. Clin. Nutr., 81(suppl):284S-291S; Tachibana et al. (2004), Nat. Struct. Mol. Biol., 11(4):380-381; and Shimizu et al. (2005), Clin. Cancer Res., 11(7):2735-2746). Moreover, it has been indicated that ECG is effective in preventing human from virus infection and in scavenging lipid free radicals. As reported by Ravindranath et al. (see Ravindranath et al. (2006), Evid Based Complement Alternat Med., 3(2):237-247), ECG has a more significant inhibitory effect against malignant tumors/cancers derived from reproductive organs, such as metastatic prostate cancer (DU145), and moderately and poorly differentiated epithelial ovarian cancers (HH450 and HH639). It has been further demonstrated that both of EGCG and ECG can inhibit the growth of BEL-7402 cells (human liver cancer cells) in a time-dependent and concentration-dependent manner and hence induce cell apoptosis, and that EGCG has a stronger inhibitory effect against BEL-7402 cells compared to ECG (see Huang et al. (2013), Journal of Guangdong Pharmaceutical University, 29(4):436-446).

CN 1732917 A discloses an antitumor composition containing active monomers from tea leaves. Specifically, the aforesaid composition comprises EGCG, ECG, EGC (epigallocatechin), EC (epicatechin), and TFG (theaflavin gallate), and has anticancer activity better than the monomers. The aforesaid composition was obtained by subjecting tea leaves to an extraction process with alcohol.

In addition, CN 1757408 A discloses a pharmaceutical composition derived from green tea, which is for treatment of cancer and tumor. CN 1757408 A further discloses a preparation method and application of such pharmaceutical composition. The aforesaid pharmaceutical composition is formulated using ingredients such as green tea leaf powder (which is green tea leaf powder derived from one or more of the following: Camellia sinensis cv. Yunnan large-leaf, Camellia sinensis cv. Jin Xuan, Camellia sinensis cv. Huangjin Gui, and Camellia sinensis cv. Yinghong-9), an extract of green tea, and isolated EGCG, ECG, EGC, EC, and TFC monomers. Compared to EGCG monomer, the aforesaid pharmaceutical composition has a stronger effect against cancer cells.

However, the purified mixtures of catechin monomers disclosed in the abovementioned patent-related documents must be prepared by subjecting tea leaves serving as a raw material to extraction, purification, and rearrangement of compositional ratio. Therefore, the preparation method is complicated, and the resulting product is pricey. Apart from the foregoing, there might be a trace of chemical substances remaining in monomers obtained via extraction and purification in a chemical manner, such that the cancer-related application of the aforesaid compositions is greatly limited. Needless to say, the examples provided in the aforesaid patent-related documents only include in vitro cell experiments, and may offer results different from those of in vivo anticancer testing.

Caffeine (1,3,7-trimethyixanthine) is also an important functional ingredient in tea leaves, and has refreshing, antioxidant, anticancer, anti-obesity, antibacterial, antidepressant, analgesic, and diuretic effects. It has been confirmed that caffeine can promote UVB irradiation-induced cell apoptosis of HaCaT (human immortalized keratinocyte cell line) cells, ensuring irreversible cancer cell damage and hence having potential ability to scavenge cancer cells (see Lei et al. (2011), Chinese Journal of Aesthetic Medicine, 20(7):1105-1107). Furthermore, as reported by Kang, caffeine extracted from coffee beans and green tea leaves can selectively hinder the activity of inositol trisphosphate receptor, and hence is able to inhibit the growth of gliomas (Kang et al. (2010), Cancer Research, 70(3):1173-1183). In addition, in recent years, it has been found that caffeine serves as an agent for enhancing the chemotherapeutic effect on tumors, can assist an anticancer drug in killing target cells, and cooperates with any of Cisplatin, paclitaxel, methotrexate, and phenobarbital to provide an synergistic effect (see Wan et al. (2003), Chinese J. Orthop., 23(3):161-164; Sun et el. (2009), Medical Recapitulate, 15(21):3297-3300; Sun et el. (2008), Orthopedic Journal of China, 16(5):376-378; and Wang et al. (2001), Chin. J. Lung Cancer, 4(3):181-183).

Han et al. have disclosed the influence of five types of tea (Hainan black tea, Hainan green tea, Fujian scented tea, Fujian Tieguanyin, and Hangzhou green tea) on N-nitrosomethylbenzylamine (NMBzA)-induced esophageal carcinoma in rats, and have found that these five types of tea had different antitumor effects when administered at the same dosage (see Han et al. (1989), Journal of Chinese Center for Disease Control and Prevention, 23(2):67-70). Specifically, Fujian Tieguanyin and Fujian scented tea had the best effect, and Hainan black tea and Hainan green tea had inferior effects. Han et al. have proposed that tea products produced from the same field have similar effects, and that the effect of tea leaves is highly relevant to the growing environment thereof.

Moreover, Ma et al. have disclosed that three types of tea, i.e. Hangzhou stir-fried tea (having a tea polyphenol content of 18.47%), Fujian scented tea (having a tea polyphenol content of 21.71%), and Fujian Tieguanyin (having a tea polyphenol content of 10.90%), have certain antioxidant activity (Ma et al. (1993), Acta Academiae Medicinae XinJian, 16(1):13-20). Fujian scented tea, which has the highest tea polyphenol content, has the strongest antioxidant effect.

Luo et al. have examined the in vitro anticancer activity of different types of tea leaves (Luo et al. (2006), Chemistry and Industry of Forest Product, 26(1):127-128). The scavenging rate of superoxide anion radical, apoptosis inducing effect, and ability to inhibit proliferation of cancer cells regarding the tested tea leaves are listed in the following descending order: Camellia sinensis cv. Yinghong-9>Camellia sinensis cv. Yunda>Camellia sinensis cv. Xiaoye>Camellia sinensis cv. Taicha. Therefore, it can be indicated that different types of tea leaves have certain degree of differences in chemical composition, as well as catechin content and ratio, and thus, the anticancer effect of tea leaves hinges on such factors.

Lastly, in most of the patent-related documents involving use of tea leaves in cancer treatment, a Chinese herbal ingredient must be used in combination. For example, CN 1158698 A discloses a tea dietary supplement comprising tea leaves, Lycium chinense, Lonicera japonica, Jasmine, chrysanthemum, Carthamus tinctorius L., Glycyrrhiza uralensis, and ginseng. The product cost of such dietary supplement is hence high due to the need of various Chinese herbal ingredients.

In view of the foregoing, the applicant deemed that it is needed to first selectively breed an antitumor/anticancer Camellia sinensis strain. By virtue of such strain, a natural tea product, which is suitable for treating cancers/tumors in human and which would not cause side effects, can be produced in a convenient manner and used solely.

### SUMMARY

Accordingly, in a first aspect, the present disclosure provides a selective breeding method of an antitumor/anticancer Camellia sinensis strain, which includes:
conducting a hybridization process so as to obtain progenies of Camellia sinensis,
subjecting the progenies of Camellia sinensis to determination of bioactive ingredient contents, wherein the determination of bioactive ingredient contents is conducted using dry leaves of the progenies of Camellia sinensis; and
selecting, from the progenies of Camellia sinensis, an antitumor/anticancer Camellia sinensis strain which meets the following five standards of bioactive ingredients: a total catechin content ≧22 wt %, an EGCG content ≧12 wt %, a catechin C content ≧2 wt %, an ECG content ≧3.5 wt %, and a caffeine content ≧4.5 wt % based on the total content of essential bioactive ingredients determined.

In a second aspect, the present disclosure provides an anti tumor/anticancer tea product, which is produced from the antitumor/anticancer Camellia sinensis strain obtained using the selective breeding method.

In a third aspect, the present disclosure provides a pharmaceutical composition comprising the antitumor/anticancer tea product.

In a fourth aspect, the present disclosure provides a supplement comprising the antitumor/anticancer tea product.

### DETAILED DESCRIPTION

It is to be understood that, if any prior art publication is referred to herein, such reference does not constitute an admission that the publication forms a part of the common general knowledge in the art, in any country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this disclosure belongs. One skilled in the art will recognize many methods and materials similar or equivalent to those described herein, which could be used in the practice of this disclosure. Indeed, this disclosure is in no way limited to the methods and materials described.

The present disclosure provides a selective breeding method of an antitumor/anticancer Camellia sinensis strain, which includes:
conducting a hybridization process so as to obtain progenies of Camellia sinensis,
subjecting the progenies of Camellia sinensis to determination of bioactive ingredient contents, wherein the determination of bioactive ingredient contents is conducted using dry leaves of the progenies of Camellia sinensis; and
selecting, from the progenies of Camellia sinensis, an antitumor/anticancer Camellia sinensis strain which meets the following five standards of bioactive ingredients: a total catechin content ≧22 wt %, an EGCG content ≧12 wt %, a catechin C content ≧2 wt %, an ECG content ≧3.5 wt %, and a caffeine content ≧4.5 wt % based on the total content of essential bioactive ingredients determined.

The term “strain” used herein may refer to a cultivar, a variety, or a subspecies of a plant species. In other words, a strain of Camellia sinensis may refer to a cultivar, a variety, or a subspecies of Camellia sinensis. Exemplary strains of Camellia sinensis include, but are not limited to, Camellia sinensis var. sinensis, Camellia sinensis var. assamica, Camellia sinensis var. dehungensis, Camellia sinensis var. pubilimba, Camellia sinensis var. waldenae, Camellia sinensis var. Huangshanzhong, a Camellia sinensis L. cultivar, and a Camellia sinensis strain obtained by hybridizing one or more of the aforesaid strains.

The term “essential bioactive ingredients” used herein refers to the bioactive ingredients normally detectable in tea leaves of a Camellia sinensis strain. The essential bioactive ingredients may include, but are not limited to, tea polyphenols, tea polysaccharides, theanine, theaflavin, lutein, folic acid, carotene, vitamin c, and Se.

The term “total catechin content” used herein refers to the total content of epigallocatechin (EGC), catechin (C), epicatechin (EC), epigallocatechin gallate (EGCG), and epicatechin gallate (ECG), and is based on the Chinese National Standard for determining catechins in tea leaves (for example, GB/T8313-2008).

As reported by Li (see Li (2006), “Synergistic Inhibition of Theaflavins against the Growth of Carcinoma Cells with Ascorbic Acid”, Zhejiang University), among catechin monomers, EGCG has the highest anticancer effect. Furthermore, ECG not only can inhibit various mutation induced by chemical carcinogenes, but also is able to suppress the mutagenesis caused by numerous carcinogenes. Catechin C is capable of entering the human body through blood circulation. Caffeine has antioxidant and antitumor effects, and is able to assist anticancer drugs in killing cancer cells. Based on the unique five standards of bioactive ingredients required by the present disclosure, the Camellia sinensis strain obtained by the selective breeding method of the present disclosure has excellent antitumor/anticancer activity due to the bioactive ingredients therein which may work synergistically.

According to the present disclosure, the hybridization process may include:
subjecting at least one paternal generation of Camellia sinensis and a maternal generation of Camellia sinensis to hybridization, wherein the paternal and maternal generations of Camellia sinensis meet at least one of the five standards of bioactive ingredients, the number of the standards which the maternal generation of Camellia sinensis meets is higher than or equal to the number of the standards which the at least one paternal generation of Camellia sinensis meets, the standards which the paternal and maternal generations of Camellia sinensis meet are in a complementary relationship.

The aforesaid complementary relationship means that: the maternal generation of Camellia sinensis and the at least one paternal generation of Camellia sinensis cooperate to meet all the five standards in a way that the standard(s) which the maternal generation of Camellia sinensis does not meet is(are) met by at least one of the at least one paternal generation of Camellia sinensis.

According to the present disclosure, the paternal and maternal generations of Camellia sinensis suitable for the aforesaid hybridization process may be strains of Camellia sinensis which have a noticeable difference(s) compared to each other, such as a noticeable difference(s) in genotype and/or phenotype.

In certain embodiments, the maternal generation of Camellia sinensis and at least one of the at least one paternal generation of Camellia sinensis are geographically distant strains which contain 25% or more of tea polyphenols on a dry matter basis of Camellia sinensis leaves.

In certain embodiments, at least one of the at least one paternal generation of Camellia sinensis is obtained by hybdridizing geographically distant strains of Camellia sinensis which contain 25% or more of tea polyphenols on a dry matter basis of Camellia sinensis leaves.

The term “geographically distant strain” refers to a strain which is to serve as a paternal or maternal generation for hybridization, and which is different in geographical position from a strain to serve as an opposite generation (the strain to serve as the opposite generation is also considered as a geographically distant strain). For example, if a strain from Fujian and a strain from a province other than Fujian are respectively selected to serve as paternal and maternal generations, these two strains are considered as geographically distant strains.

According to the present disclosure, the hybridization process may further include:
obtaining seedlings from offsprings of Camellia sinensis produced by the hybridization of the paternal and maternal generations of Camellia sinensis and sowing the seedlings after ripening of fruits of the offsprings of Camellia sinensis; and
subjecting the seedlings to transplantation after sowing.

In certain embodiments of the present disclosure, each of the seedlings, when growing to have a height of equal to and higher than 35 cm, is transplanted.

According to the present disclosure, the hybridization process may be conducted using a conventional technique. In some embodiments, the hybridization process may be conducted using artificial pollination or bee pollination. In certain embodiments, the hybridization process may be simple hybridization (i.e. hybridization of one maternal generation and one paternal generation) or multi-paternal hybridization (i.e. hybridization of one maternal generation and two or more paternal generations).

The paternal and maternal generations of Camellia sinensis may be cultivated in a field that has a relative humidity of higher than or equal to 90%. In certain embodiments, the field is at an elevation of lower than or equal to 300 meters above sea level, and receives, compared to normal fields, 550-650 Kg more of potassium sulfate, 300-400 Kg more of calcium superphosphate, and 8500-9500 Kg more of fresh stems and leaves of Cassia obtusifolia L. per hectare each year.

The term “normal field” refer to a field which receives about 150 kg of nitrogen per hectare each year, and the nitrogen-phosphorus-potassium ratio of which ranges from 2:1:1 to 4:1:1 (the definition of such term can be found in Luo (2008), Tea Tree Cultivation, page 235, China Agriculture Press).

The geographically distant strain of Camellia sinensis which contains 25% or more of tea polyphenols and the progeny thereof can be selected from the following examples: strains from Yunnan province (e.g. Camellia sinensis cv. Gongnong, Camellia sinensis cv. Haigong, Camellia sinensis cv. Bingdao large-leaf, Camellia sinensis cv. Mengku, Yunkang No. 10, and Camellia sinensis cv. Baohong), strains form Fujian province (e.g. Camellia sinensis cv. Baxian, Camellia sinensis cv. Meizhang, Camellia sinensis cv. Yingchun, Camellia sinensis cv. Chunlan, 1005, DC-1, HLG-2, MLC-3), strains from Hubei province (e.g. Camellia sinensis cv. E-Cha No. 5), strains from Hunan province (e.g. Camellia sinensis cv. Xiangcha No. 1), strains from Guangdong province (e.g. Camellia sinensis cv. Lechang Baimao, Camellia sinensis cv. Hongyin, and Camellia sinensis cv. Yinghong-9), strains from Guangxi province (e.g. Camellia sinensis cv. Heng County small-leaf), and strains from Hainan province (e.g. Camellia sinensis cv. Hainan large-leaf).

According to the present disclosure, terminal buds of the seedlings growing to a height ranging from 20 cm to 30 cm may be removed after sowing and before transplantation. In some embodiments, each of the seedlings growing to have a height of equal to and higher than 35 cm is transplanted during a period of time spanning from November to subsequent February.

In some embodiments, the progenies of Camellia sinensis obtained after cultivation of the seedlings for three years are subjected to the determination of bioactive ingredient contents for three consecutive years, and each of the progenies of Camellia sinensis which meets the five standards of bioactive ingredients for three consecutive years is the antitumor/anticancer Camellia sinensis strain. In an exemplary embodiment, each of the progenies of Camellia sinensis which meets the five standards of bioactive ingredients in the same harvest season for three consecutive years is the antitumor/anticancer Camellia sinensis strain.

The present disclosure further provides an antitumor/anticancer tea product produced from the antitumor/anticancer Camellia sinensis strain obtained using the aforesaid selective breeding method.

According to the present disclosure, tea leaves of the aforesaid antitumor/anticancer Camellia sinensis strain may be subjected to plucking according to the one-bud-one-tip method (which means that young, green tea leaves are to be plucked) after the aforesaid antitumor/anticancer Camellia sinensis strain grows to meet the five standards of bioactive ingredients, so as to serve as a starting material for producing the antitumor/anticancer tea product. In some embodiments, heating is conducted after the plucking step for enzyme deactivation or drying. A first heating step may be conducted after the plucking step for the purpose of enzyme deactivation, and may include rapidly raising the temperature of the surface of the tea leaf to 80° C. or higher and maintaining such temperature for 6 minutes. A second heating step may be conducted after the first heating step for the purpose of drying, and may include further raising the temperature of the surface of the tea leaf to 90° C. and maintaining such temperature until the water content is lower than 5%.

According to the present disclosure, the antitumor/anticancer tea product is suitable for treating a cancer/tumor selected from the group consisting of liver cancer, ovarian cancer, cervical cancer, lung cancer, glioblastoma, mammary gland cancer, prostate cancer, leukemia, gastric cancer, colon cancer, squamous cell carcinoma, and pancreatic cancer.

Still further, the present disclosure provides a pharmaceutical composition and a supplement, each of which comprises the aforesaid antitumor/anticancer tea product. The pharmaceutical composition is suitable for treating a cancer/tumor selected from the group consisting of liver cancer, ovarian cancer, cervical cancer, lung cancer, glioblastoma, mammary gland cancer, prostate cancer, leukemia, gastric cancer, colon cancer, squamous cell carcinoma, and pancreatic cancer.

The present disclosure will be further described in the following examples. However, it should be understood that the following examples are solely intended for the purpose of illustration and should not be construed as limiting the present disclosure in practice.

### EXAMPLES

### General Experimental Procedures

### 1. Determination of Bioactive Ingredient Contents

Fresh leaves of a respective Camellia sinensis strain were subjected to plucking according to the one-bud-one-tip method, followed by heating under a high temperature. After the plucking step, for enzyme deactivation, the temperature of the surfaces of the tea leaves was rapidly raised to 80° C. or higher, and such temperature was maintained for 6 minutes. Subsequently, for drying, the temperature of the surfaces of the tea leaves was raised to 90° C., and such temperature was maintained until the water content was lower than 5%. The contents of the essential bioactive ingredients in the resulting dry leaves of Camellia sinensis were determined generally according to the the following Chinese National Standards or via conventional techniques: GB/T8313-2008 (for determining the contents of tea polyphenols, catechins, and caffeine in tea leaves), anthrone-sulfuric acid assay (for determining the content of tea polysaccharides), GB/T 8314-2002 (for determining the content of free amino acids in tea), GB/T 30483-2013 (for determining the content of theaflavins in tea leaves), high performance liquid chromatography (which is for determining the content of xanthophylls, and which was conducted using a Spherisorb C₁₈ column (5 μm, 250 mm×4.6 mm), as well as a mobile phase composed of acetonitrile:methanol (85:15) and methanol:ethyl acetate(68:32), under linear gradient elution and detection at 445 nm), GB/T 5009.211-2008 (for determining the content of folic acid in food), GB/T 5009.83-2003 (for determining the content of carotenes in food), GB/T 6195-86 (for determining the content of vitamin c in fruits and vegetables via titration with 2,6-dichlorophenolindophenol), and GB/T 21729-2008 (for determining the content of selenium in tea leaves). In other words, the essential bioactive ingredients detected were tea polyphenols, tea polysaccharides, theanine, theaflavin, xanthophylls, folic acid, carotene, vitamin c, and Se.

The following five standards were applied to select an antitumor/anticancer Camellia sinensis strain, as well as paternal and maternal generations of Camellia sinensis for hybridization: a total catechin content ≧22 wt %, an EGCG content ≧12 wt %, a catechin C content ≧2 wt %, an ECG content ≧3.5 wt %, and a caffeine content ≧4.5 wt %, based on the total content of essential bioactive ingredients determined.

2. Preparation of Tea Product of Camellia sinensis

The dry tea leaves of the respective Camellia sinensis strain obtained according to the method described in section 1 above were ground into fine powder, followed by brewing with boiling water. Filtration was conducted, followed by precipitation. The resulting supernatant was collected to serve as a tea product.

### Example 1. Selective Breeding of Antitumor/Anticancer Camellia sinensis Strains

Two antitumor/anticancer Camellia sinensis strains were prepared in this example and are respectively referred to as Camellia sinensis Strains 1 and 2 hereinafter. Selective breeding of Camellia sinensis Strain 1 is described first below.

DC-1, which contained a high amount of tea polyphenols (about 15% to 28% of tea polyphenols), which was obtained by natural hybridization, and which met the standards for total catechin content, ECG content, catechin C content, and caffeine content as described in section 1 of “General Experimental Procedures”, was used as a maternal generation of Camellia sinensis. Furthermore, Camellia sinensis cv. Baxian (which met the standard for ECG content as described in section 1 of “General Experimental Procedures”, and which has a germplasm certificate number GS13012-1994 issued by Chinese Crop Germplasm Information System (CGRIS)) and 1005 which is a strain of Camellia sinensis produced through hybridization of geographically distant strains (1005 met the standards for EGCG content and ECG content as described in section 1 of “General Experimental Procedures”) were used as paternal generations of Camellia sinensis. DC-1, Camellia sinensis cv. Baxian, and 1005 were subjected to multi-paternal hybridization using artificial pollination.

After ripening of fruits of the offsprings of Camellia sinensis obtained by virtue of the aforesaid multi-paternal hybridization, seedlings were obtained from the offsprings of Camellia sinensis and were subjected to sowing. When the seedlings grew to have a height of 25 cm, the terminal buds of the seedlings were removed. During the period of time spanning from November to subsequent February, the seedlings grew to have a height of 35 cm were subjected to transplantation.

The progenies of Camellia sinensis obtained after cultivation of the seedlings for three years were subjected to determination of bioactive ingredient contents for three consecutive years in each season generally according to the method described in section 1 of “General Experimental Procedures”, so as to determine whether each of the progenies of Camellia sinensis met the five standards of bioactive ingredients as described in section 1 of “General Experimental Procedures”. The progenies of Camellia sinensis obtained after cultivation of the seedlings for three years had the following contents of essential bioactive ingredients: more than 20 wt % of tea polyphenols, 1 wt % to 2 wt % of tea polysaccharides, 1 wt % to 2 wt % of theanine, 1 wt % to 5.9 wt % of theaflavin, 0.14 wt % to 0.80 wt % of lutein, 0.0005 wt % to 0.0007 wt % of folic acid, 0.06 wt % of carotene, 0.6 mg to 2.5 mg of vitamin c, and 0.001 mg of Se, based on the weight of dry Camellia sinensis leaves determined. Among the progenies of Camellia sinensis, a strain which met all the five standards of bioactive ingredients as described in section 1 of “General Experimental Procedures” for three consecutive year served as Camellia sinensis Strain 1.

Camellia sinensis Strain 2 was produced substantially according to the selective breeding method of Camellia sinensis Strain 1, except that MLC-3 (i.e. a strain which met the standards for total catechin content, EGCG content, catechin C content, and ECG content as described in section 1 of “General Experimental Procedures”) was used as the maternal generation of Camellia sinensis, and that Camellia sinensis cv. Haigong of Yunnan province (which met the standards for ECG content and catechin C content as described in section 1 of “General Experimental Procedures”), Camellia sinensis cv. Baohong (which met the standards for ECG content and caffeine content as described in section 1 of “General Experimental Procedures”), and HLG-2 (i.e. a strain which met the standards for total catechin content, caffeine content, and EGCG content as described in section 1 of “General Experimental Procedures”) were used as the paternal generations of Camellia sinensis. DC-1, 1005, MLC-3, and HLG-2 were provided by Agricultural Product Quality Institute at Fujian Agriculture and Forestry University.

### Example 2. Comparison Between Bioactive Ingredients of Camellia sinensis Strains of Present Disclosure and Conventional Camellia sinensis Strain

In order to compare the bioactive ingredients of Camellia sinensis Strains 1 and 2 with those of a conventional Camellia sinensis strain, a conventional Camellia sinensis strain (which is referred to as FuYun no. 6, which was provided by Agricultural Product Quality Institute at Fujian Agriculture and Forestry University, and which has a germplasm certificate number GS13033-1987 issued by CGRIS) was subjected to determination of bioactive ingredients according to the methods described in section 1 of “General Experimental Procedures”. The contents of caffeine and catechins in Camellia sinensis Strains 1 and 2 and the conventional Camellia sinensis strain are shown in Table 1 below.

TABLE I
Contents of caffeine and catechins (wt %) in Camellia
sinensis Strains 1 and 2 and conventional Camellia
sinensis strain

| | | | | | | | | | | Total |
|---|---|---|---|---|---|---|---|---|---|---|
| Strain | GC | EGC | C | Caffeine | EC | EGCG | GCG | ECG | CG | catechins |
| Camellia | 0.45 | 1.85 | 3.00 | 5.38 | 0.64 | 13.42 | 0.49 | 3.58 | 0.30 | 22.49 |
| sinensis | | | | | | | | | | |
| Strain 1 | | | | | | | | | | |
| Camellia | 0.52 | 3.17 | 2.03 | 4.99 | 1.27 | 12.68 | 0.25 | 4.18 | 0.45 | 23.33 |
| sinensis | | | | | | | | | | |
| Strain 2 | | | | | | | | | | |
| Conventional | 0.15 | 2.78 | 0.06 | 2.61 | 1.19 | 6.07 | 0.18 | 2.36 | 0.09 | 12.46 |
| Camellia | | | | | | | | | | |
| sinensis | | | | | | | | | | |
| strain | | | | | | | | | | |

GC: gallocatechin
CG: catechin-3-gallate
GCG: gallocatechin gallate
Total catechins: EGC + C + EC + EGCG + ECG

As shown in Table 1, generally speaking, Camellia sinensis Strains 1 and 2 have higher contents of the bioactive ingredients effective in the treatment of tumors/cancers compared to the conventional Camellia sinensis strain. The applicant hence deduced that the Camellia sinensis strain according to the present disclosure may have satisfactory antitumor/anticancer activity and may be used to prepare antitumor/anticancer tea products.

### Example 3. In Vitro Testing for Antitumor/Anticancer Activity of Tea Product Prepared from Camellia sinensis Strain of Present Disclosure

This example was conducted to determine the antitumor/anticancer activities of the Camellia sinensis strain according to the present disclosure.

A. Preparation of Antitumor/Anticancer Tea Product of Present Disclosure

Tea leaves of Camellia sinensis Strain 1 obtained in Example 1 and the conventional Camellia sinensis strain used in Example 2 were respectively used to prepare Tea Product 1 and Conventional Tea Product according to the method described in section 2 of “General Experimental Procedures”.

B. Preparation of Test Tea Samples from Tea Products and Preparation of Comparative Test Samples from Commercially Available Agents

Each of Tea Product 1 and Conventional Tea Product obtained in section A of this example was used to prepare test tea samples according to the method described below. A proper amount of the respective tea product was added into sterile water to prepare a stock solution with a concentration of 100 mg/mL, followed by using a membrane filter with a pore size of 0.22 μm to conduct vacuum filtration and sterilization. The resulting stock solution was diluted with a respective complete medium shown in Table 3 below to form test tea samples with different concentrations. In other words, the thus obtained test tea samples to be used for treating a respective cancer/tumor cell line shown in Table 3 were prepared using the same complete medium as that for the respective cancer/tumor cell line.

The test tea samples prepared from Tea Product 1 respectively had concentrations of 0.1 μg/mL, 0.05 μg/mL, 0.01 μg/mL, 0.025 μg/mL, 0.0125 μg/mL, 0.00625 μg/mL, 0.005 μg/mL, 0.003125 μg/mL, 0.0025 μg/mL, 0.001563 μg/mL, 0.00125 μg/mL, 0.000781 μg/mL, 0.000625 μg/mL, 0.000417 μg/mL, 0.000391 μg/mL, 0.000313 μg/mL, 0.0003125 μg/mL, 0.000195 μg/mL, 0.000139 μg/mL, 9.77×10⁻⁵ μg/mL, 4.63×10⁻⁵ μg/mL, and 1.54×10⁻⁵ μg/mL. The test tea samples prepared from Conventional Tea Product respectively had concentrations of 0.1 μg/mL, 0.05 μg/mL, 0.01 μg/mL, 0.025 μg/mL, 0.0125 μg/mL, 0.00625 μg/mL, 0.005 μg/mL, 0.003125 μg/mL, 0.0025 μg/mL, 0.001563 μg/mL, 0.00125 μg/mL, and 0.000625 μg/mL.

Moreover, the following commercially available agents were used to prepare comparative test samples: Cisplatin (Faulding), Epirubicin (Pfizer Pharmaceutical Co., Ltd., Wuxi, China), Etoposide (Qilu Pharmaceutical Co., Ltd., China), 5-fluorouracil injection (Shanghai Xudong Haipu Pharmaceutical Co., Ltd., Shanghai, China), Gefitinib (AstraZeneca UK Ltd.), Glivec (Novartis Pharma Stein AG), and EGCG monomer (having a purity of 95% and obtained from Sigma-Aldrich).

Specifically, each of the aforesaid commercially available agents was subjected to the method described below to prepare comparative test samples. A proper amount of a respective commercially available agent was dissolved in phosphate-buffered saline (PBS) to prepare a stock solution with a concentration of 5 mg/mL, followed by using a membrane filter with a pore size of 0.22 μm to conduct vacuum filtration and sterilization. The resulting stock solution was diluted with a respective complete medium shown in Table 3 below to form comparative test samples with different concentrations. To be specific, the comparative test samples prepared from the respective commercially available agent have the concentrations shown in Table 2 below. The thus obtained comparative test samples to be used for treating a respective cancer/tumor cell line shown in Table 3 were prepared using the same complete medium as that for the respective cancer/tumor cell line.

TABLE 2
Concentrations of comparative test samples prepared
from commercially available agents

| | Concentrations |
|---|---|
| EGCG | 640 μg/mL, 320 μg/mL, 160 μg/mL, 80 μg/mL, |
| monomer test | 40 μg/mL, 20 μg/mL, 10 μg/mL, 5 μg/mL, 2.5 μg/mL, |
| samples | 1.25 μg/mL, 0.625 μg/mL and 0.3125 μg/mL |
| 5-fluorouracil | 80 μg/mL, 40 μg/mL, 20 μg/mL, 10 μg/mL, 5 μg/mL, |
| test samples | 2.5 μg/mL, 1.25 μg/mL, 0.625 μg/mL, 0.3125 μg/mL, |
| | 0.15625 μg/mL, 0.078125 μg/mL, |
| | 0.039063 μg/mL, 0.019531 μg/mL |
| Cisplatin test | 16 μg/mL, 8 μg/mL, 4 μg/mL, 2 μg/mL, 1 μg/mL, |
| samples | 0.5 μg/mL, 0.25 μg/mL, 0.125 μg/mL, and 0.0625 μg/mL |
| Epirubicin test | 4 μg/mL, 2 μg/mL, 1 μg/mL, 0.5 μg/mL, 0.25 μg/mL, |
| samples | 0.125 μg/mL, 0.0625 μg/mL, 0.03125 μg/mL, |
| | and 0.015625 μg/mL |
| Etoposide test | 32 μg/mL, 16 μg/mL, 8 μg/mL, 4 μg/mL, 2 μg/mL, |
| samples | 1 μg/mL, 0.5 μg/mL, 0.25 μg/mL, and 0.125 μg/mL |
| Glivec test | 0.5897 μg/mL, 0.2949 μg/mL, 0.1474 μg/mL, |
| samples | 0.0737 μg/mL, 0.0369 μg/mL, and 0.0184 μg/mL |
| Gefitinib test | 14.3008 μg/mL, 7.1504 μg/mL, 3.5752 μg/mL, |
| samples | 1.7876 μg/mL, 0.8938 μg/mL, and 0.4469 μg/mL |

### C. In Vitro Antitumor/Anticancer Testing

The following cancer/tumor cells purchased from Chinese Academy of Sciences, Shanghai Institutes for Biological Sciences, Institute of Biochemistry and Cell Biology were used for in vitro antitumor/anticancer testing: human liver cancer cell line HepG2, human ovarian cancer cell line SK-OV-3, human cervical cancer cell line Hela, human lung cancer cell line A549, human glioblastoma cell line U-87MG, human mammary gland/duct cancer cell line BT-474, human prostate cancer cell line PC-3, human leukemia cell line HL-60, human gastric cancer cell line SMMC-7721, human colon cancer cell line HCT-116, human squamous cell carcinoma cell line A431, human leukemia cell line K562, human pancreatic cancer cell line PANC-1, and human gastric cancer cell line BGC-823. These cancer/tumor cells were respectively cultivated using the complete mediums shown in Table 3 below.

TABLE 3
Mediums for cultivation of cancer/tumor cells

| Cell line | Complete mediums |
|---|---|
| SK-OV-3 | McCoy's 5A (Invitrogen) + 10% fetal bovine serum |
| | (FBS) (Cat. No. 10099-141, GIBCO) |
| Hela | Eagle's Minimum Essential Medium |
| | (EMEM) (Invitrogen) + 1 mM sodium pyruvate + |
| | 1.5 g/L, NaHCO₃ + 10% FBS |
| A549 | F12 K (modification of Ham's F-12 Nutrient |
| | Mixture) (Invitrogen) + 10% FBS |
| U-87MG | EMEM + 1 mM sodium pyruvate + 1.5 g/L |
| | NaHCO₃ + 10% FBS |
| BT-474 | Roswell Park Memorial Institute (RPMI) 1640 |
| | (Invitrogen) + 10% FBS |
| PC-3 | F12K + 10% FBS |
| HL-60 | Iscove's Modified Dulbecco's Medium (IMDM) |
| | (Invitrogen) + 20% FBS |
| SMMC-7721, | 90% RPMI 1640 + 10% FBS |
| K562, BGC-823 | |
| HCT-116, A431, | Dulbecco's Modified Eagle's medium |
| HepG2, PANC-1 | (DMEM)(Invitrogen) + 10% FBS |

The cultivation of each cancer/tumor cell line is described as follows. The cancer/tumor cancer cells were cultivated in the respective complete medium supplemented with 100 μg/mL penicillin and 100 μg/mL streptomycin in an incubator (37° C., 100% relative humidity, 5% CO₂). When adherent cancer/tumor cells reached 80% confluence during the log-phase, cell detachment was conducted using 0.6 mL of 0.3% trypsin. Afterwards, a cell suspension having a cell density of 1.0×10⁴ cells/mL was prepared. 100 μL of the cell suspension (i.e. 1000 cancer/tumor cells) was added into wells of a 96-well plate (Cat. No. 3599, Corning Costar), followed by cultivation in an incubator (37° C., 100% relative humidity, 5% CO₂) for 24 hours.

As experimental groups, the tumor/cancer cells of each cell line were treated with the test tea samples prepared from one or more of the tea products and the comparative test samples prepared from one or more of the commercially available agents as obtained in section B of this example (for more detailed information about the tea product(s) and the commercially available product (s) used to treat each cell line, please see Table 4 below). To be specific, 6 to 10 test tea samples prepared from the same tea product and having different concentrations, as well as 6 to 10 comparative test samples prepared from the same commercially available agent and having different concentrations, were utilized to treat the tumor/cancer cells of each cell line. For each experimental group, 6 repetitions of tumor/cancer treatment were performed. For the control group, 100 μL of PBS was added to a well containing the medium and the cancer/tumor cells of the respective cell line. For the blank group, 100 μL of PBS was added to a well containing a complete medium the same as that used for cultivation of the cancer/tumor cells of the respective cell line.

TABLE 4
Tea product(s) and commercially available product(s)
used to treat each cell line

| Cell line | Test substances |
|---|---|
| SK-OV-3, Hela, A549, U-87MG | Tea product 1, EGCG monomer, |
| | Cisplatin |
| BT-474, PC-3 | Tea product 1, EGCG monomer, |
| | Epirubicin |
| HL-60 | Tea product 1, EGCG monomer, |
| | Etoposide |
| SMMC-7221, HCT116, A431, | Tea product 1, EGCG monomer, |
| BGC-823 | 5-fluorouracil |
| PANC-1 | Tea product 1, Gefitinib |
| K562 | Tea product 1, Glivec |
| HepG2 | Tea product 1, Conventional Tea |
| | Product, 5-fluorouracil |

After the treatment described above, cultivation was conducted in an incubator (37° C., 100% relative humidity, 5% CO₂) for 48 hours. Subsequently, the liquid containing the medium and the sample in the well was removed, and a respective complete medium (shown in Table 3 above) containing 10% CCK-8 (Cell Counting Kit-8)(Dojindo Molecular Technologies, Inc., Cat. No. CK04-13) was added. Cultivation was performed in an incubator (37° C.) for 2 to 4 hours, followed by gently shaking. For the resulting culture, absorbance at 450 nm and absorbance at 650 nm (serving as a reference) were measured using SpectraMax) M5 Microplate Reader (Molecular Devices, LLC.). The percent growth inhibition (%) of the tumor/cancer cells of the respective cell line was calculated using the following Equation I:

A=[(C−B)/(C−D)]λ100% (I)
A=percent growth inhibition (%)
B=Optical absorbance (OA) of respective experimental group (i.e., culture containing CCK-8 and cancer/tumor cells treated with respective test substance)
C=OA of control group (i.e. culture containing CCK-8 and cancer/tumor cells)
D=OA of blank group (i.e. culture containing CCK-8 and respective complete medium)
   For each of the tea products and the commercially available agents, the thus obtained values of percent growth inhibition at different concentrations were used to determine the half maximal inhibitory concentration (IC50) according to the conventional Bliss method.

### Results:

The inhibitory effect of each test substance on the proliferation of the respective cancer/tumor cell line is shown in Tables 5 to 14 below. IC50<30 μg/mL indicates that the tea product of the present disclosure exhibit an in vitro killing activity against cancer/tumor cells.

First of all, the inhibitory effects of Tea Product 1, Conventional Tea Product, and 5-fluorouracil on the proliferation of human liver cancer cell line HepG2 are discussed (see Tables 5 and 6).

TABLE 5
Inhibitory effects of Tea Product 1, Conventional
Tea Product, and 5-fluorouracil on proliferation
of human liver cancer cell line HepG2

| | | Test tea | | | |
|---|---|---|---|---|---|
| | | samples prepared | | | |
| Test tea samples prepared | | from Conventional Tea | | | |
| from Tea Product 1 | | Product | | 5-fluorouracil test samples | |
| | Percent | | Percent | | Percent |
| | growth | | growth | | growth |
| Concentrations | inhibition | Concentrations | inhibition | Concentrations | inhibition |
| (μg/mL) | (%) | (μg/mL) | (%) | (μg/mL) | (%) |
| 0.1 | 82.50 | 0.1 | 70.51 | 5 | 89.13 |
| 0.05 | 84.92 | 0.05 | 74.54 | 2.5 | 85.97 |
| 0.025 | 76.19 | 0.025 | 75.29 | 1.25 | 83.28 |
| 0.0125 | 41.47 | 0.0125 | 72.96 | 0.625 | 63.69 |
| 0.00625 | 33.99 | 0.00625 | 18.72 | 0.3125 | 37.54 |
| 0.003125 | 29.31 | 0.003125 | −2.29 | 0.15625 | 24.51 |
| 0.001563 | 38.48 | 0.001563 | −31.31 | 0.078125 | 10.93 |

| | | |
|---|---|---|
| IC50 (μg/mL) = 0.0094 | IC50 (μg/mL) = 0.2252 | IC50 (μg/mL) = 0.4574 |

As shown in Table 5, Tea Product 1 has an evident in vitro inhibitory effect against the proliferation of HepG2 cells (IC50 (μg/mL)=0.0094), and such effect is generally concentration-dependent. Furthermore, the highest percent growth inhibition of Tea Product 1 (84.92%) was achieved when the concentration of Tea Product 1 was 0.05 μg/mL. Therefore, the inhibitory effect of Tea Product 1 is superior to that of Conventional Tea Product (the highest percent growth inhibition of Conventional Tea Product is 75.29%), and is matchable with that of the chemotherapy drug 5-fluorouracil (the highest percent growth inhibition of 5-fluorouracil (89.13%) was achieved when the concentration of 5-fluorouracil was 5 μg/mL).

TABLE 6
Inhibitory effects of Tea Product 1 and Conventional
Tea Product on proliferation of human liver cancer
cell line HepG2

| | |
|---|---|
| | Test tea samples prepared |
| Test tea samples prepared | from Conventional Tea |
| from Tea Product 1 | Product |

| | Percent | | Percent |
|---|---|---|---|
| | growth | | growth |
| Concentrations | inhibition | Concentrations | inhibition |
| (μg/mL) | (%) | (μg/mL) | (%) |
| 0.01 | 68.12 | 0.01 | 55.71 |
| 0.005 | 62.65 | 0.005 | 29.53 |
| 0.0025 | 42.87 | 0.0025 | 19.14 |
| 0.00125 | 29.80 | 0.00125 | 20.03 |
| 0.000625 | 18.82 | 0.000625 | 2.57 |

| | |
|---|---|
| IC50 (μg/mL) = 0.00310 | IC50 (μg/mL) = 0.00630 |

Further referring to Table 6, it can be demonstrated again that the tea product according to the present disclosure is more effective in inhibiting cancer/tumor cells compared to the conventional tea product.

Turning to the inhibitory effects of Tea Product 1 and the commercially available agents on the proliferation of other cancer/tumor cell lines, please refer to Tables 7 to 14 below.

TABLE 7
Inhibitory effects of Tea Product 1, EGCG monomer,
and 5-fluorouracil on proliferation of human colon
cancer cell line HCT-116 and human squamous cell
carcinoma cell line A431

| | Test tea samples prepared | | | | | |
|---|---|---|---|---|---|---|
| | from Tea Product 1 | | EGCG monomer test samples | | 5-fluorouracil test samples | |
| | | Percent | | Percent | | Percent |
| | | growth | | growth | | growth |
| Cell | Concentrations | inhibition | Concentrations | inhibition | Concentrations | inhibition |
| line | (μg/mL) | (%) | (μg/mL) | (%) | (μg/mL) | (%) |
| HCT-116 | 0.025 | 99.64 | 80 | 66.98 | 80 | 99.49 |
| | 0.0125 | 59.55 | 40 | 34.52 | 40 | 97.87 |
| | 0.00625 | 34.63 | 20 | −1.58 | 20 | 95.85 |
| | 0.003125 | 2.64 | 10 | −4.12 | 10 | 91.89 |
| | 0.001563 | −2.90 | 5 | −7.91 | 5 | 89.94 |
| | 0.000731 | 16.51 | 2.5 | 10.74 | 2.5 | 88.54 |
| | 0.000391 | 13.72 | | | 1.25 | 36.38 |
| | 0.000195 | 22.59 | | | 0.625 | 32.39 |
| | 9.77 × 10⁻⁵ | 37.55 | | | 0.3125 | 70.14 |
| | IC50 (μg/mL) = 0.0086 | | IC50 (μg/mL) = 55.72 | | IC50 (μg/mL) = 0.0493 | |
| A431 | 0.025 | 96.84 | 80 | 30.13 | 80 | 98.75 |
| | 0.0125 | 65.59 | 40 | 55.14 | 40 | 98.15 |
| | 0.00625 | 38.73 | 20 | 34.07 | 20 | 96.33 |
| | 0.003125 | 25.45 | 10 | 24.03 | 10 | 33.66 |
| | 0.001563 | 21.65 | 5 | 19.18 | 5 | 84.36 |
| | 0.000781 | 14.55 | 2.5 | 12.70 | 2.5 | 82.99 |
| | 0.000391 | 8.78 | 1.25 | 10.00 | 1.25 | 83.06 |
| | 0.000195 | 10.98 | 0.625 | 13.31 | 0.625 | 83.52 |
| | 9.77 × 10⁻⁵ | 5.41 | 0.3125 | 7.79 | 0.3125 | 82.36 |

| | | |
|---|---|---|
| IC50 (μg/mL) = 0.0066 | IC50 (μg/mL) = 29.51 | IC50 (μg/mL) = 0.2721 |

TABLE 8
Inhibitory effects of Tea Product 1, EGCG monomer
(having a purity of 95%), and 5-fluorouracil on
proliferation of human gastric cancer cell line
SMMC-7721 and human gastric cancer cell line
BGC-823

| | Test tea samples prepared | | | | | |
|---|---|---|---|---|---|---|
| | from Tea Product 1 | | EGCG monomer test samples | | 5-fluorouracil test samples | |
| | | Percent | | Percent | | Percent |
| | | growth | | growth | | growth |
| | Concentrations | inhibition | Concentrations | inhibition | Concentrations | inhibition |
| Cell line | (μg/Ml) | (%) | (μg/mL) | (%) | (μg/mL) | (%) |
| SMMC-771 | 0.025 | 74.17 | 640 | 97.44 | 80 | 90.95 |
| | 0.0125 | 12.97 | 320 | 98.50 | 40 | 80.18 |
| | 0.00625 | 2.21 | 160 | 92.58 | 20 | 79.08 |
| | 0.003125 | −2.91 | 80 | 40.85 | 10 | 81.58 |
| | 0.001563 | −3.12 | 40 | 6.33 | 5 | 84.29 |
| | 0.000781 | −0.69 | 20 | 1.33 | 2.5 | 82.73 |
| | 0.000391 | −3.45 | 10 | 3.45 | 1.25 | 80.97 |
| | 0.000195 | 1.36 | 5 | 3.31 | 0.625 | 62.43 |
| | 9.77 × 10⁻⁵ | −2.83 | 2.5 | 4.93 | 0.3125 | 28.55 |
| | IC50 (μg/mL) = 0.0195 | | IC50 (μg/mL) = 77.41 | | IC50 (μg/mL) = 0.383 | |
| BGC-823 | 0.1 | 99.39 | 640 | 97.03 | 80 | 62.75 |
| | 0.05 | 99.79 | 320 | 98.16 | 40 | 52.74 |
| | 0.025 | 95.39 | 160 | 97.87 | 20 | 40.90 |
| | 0.0125 | 43.54 | 80 | 60.53 | 10 | 23.86 |
| | 0.00625 | −0.75 | 40 | 28.02 | 5 | 11.58 |
| | 0.003125 | −9.40 | 20 | 6.47 | 2.5 | 0.92 |
| | 0.001563 | −6.41 | 10 | −0.64 | 1.25 | −1.41 |
| | 0.000781 | −3.79 | 5 | −1.06 | 0.625 | −4.99 |
| | 0.000391 | −1.35 | 2.5 | −0.82 | 0.3125 | −5.82 |

| | | |
|---|---|---|
| IC50 (μg/mL) = 0.0159 | IC50 (μg/mL) = 62.94 | IC50 (μg/mL) = 2.5467 |

TABLE 9
Inhibitory effects of Tea Product I, EGCG monomer
(having a purity of 95%), and Cisplatin on
proliferation of human ovarian cancer cell line
SK-OV-3 and human cervical cancer cell line Hela

| | Test tea samples prepared | | | | | |
|---|---|---|---|---|---|---|
| | from Tea Product 1 | | EGCG monomer test samples | | Cisplatin test samples | |
| | | Percent | | Percent | | Percent |
| | | growth | | growth | | growth |
| Cell | Concentrations | inhibition | Concentrations | inhibition | Concentrations | inhibition |
| line | (μg/mL) | (%) | (μg/mL) | (%) | (μg/mL) | (%) |
| SK-OV-3 | 0.025 | 99.53 | 80 | 99.42 | 16 | 83.65 |
| | 0.0125 | 99.77 | 40 | 81.73 | 8 | 88.36 |
| | 0.00625 | 83.56 | 20 | 36.88 | 4 | 81.38 |
| | 0.003125 | 46.28 | 10 | 4.47 | 2 | 65.87 |
| | 0.001563 | 9.56 | 5 | 6.15 | 1 | 51.92 |
| | 0.000781 | 5.18 | 2.5 | 5.54 | 0.5 | 28.16 |
| | 0.000391 | 1.07 | 1.25 | 3.64 | 0.25 | 13.36 |
| | 0.000195 | 4.60 | 0.625 | 3.98 | 0.125 | 6.86 |
| | 9.77 × 10⁻⁵ | −0.50 | 0.3125 | 2.20 | 0.0625 | 5.63 |
| | IC50 (μg/mL) = 0.0033 | | IC50 (μg/mL) = 24.05 | | IC50 (μg/mL) = 1.112 | |
| Hela | 0.025 | 93.22 | 80 | 36.66 | 16 | 98.98 |
| | 0.0125 | 14.06 | 40 | −23.80 | 8 | 98.3 |
| | 0.00625 | −5.09 | 20 | −20.22 | 4 | 90.1 |
| | 0.003125 | −4.62 | 10 | −22.63 | 2 | 16.62 |
| | 0.001563 | −2.29 | 5 | −6.50 | 1 | −29.75 |
| | 0.000781 | 4.81 | 2.5 | −8.94 | 0.5 | −33.69 |
| | 0.000391 | 2.98 | 1.25 | 1.08 | 0.25 | −11.93 |
| | 0.000195 | 0.46 | 0.625 | −0.68 | 0.125 | −7.31 |
| | 9.77 × 10⁻⁵ | −0.59 | 0.3125 | 4.13 | 0.0625 | −3.05 |

| | | |
|---|---|---|
| IC50 (μg/mL) = 0.0166 | IC50 (μg/mL) = 101.2 | IC50 (μg/mL) = 2.656 |

TABLE 10
Inhibitory effects of Tea Product 1, EGCG monomer
(having a purity of 95%), and Cisplatin on
proliferation of human lung cancer cell line A549
and human glioblastoma cell line U-87MG

| | Test tea samples prepared | | | | | |
|---|---|---|---|---|---|---|
| | from Tea Product 1 | | EGCG monomer test samples | | Cisplatin test samples | |
| | | Percent | | Percent | | Percent |
| | | growth | | growth | | growth |
| Cell | Concentrations | inhibition | Concentrations | inhibition | Concentrations | inhibition |
| line | (μg/mL) | (%) | (μg/mL) | (%) | (μg/mL) | (%) |
| A549 | 0.025 | 99.64 | 80 | 60.18 | 16 | 97.45 |
| | 0.0125 | 67.69 | 40 | 35.39 | 3 | 88.98 |
| | 0.0062 | 34.92 | 20 | 23.21 | 4 | 57.57 |
| | 0.003125 | 15.36 | 10 | 9.62 | 7 | 36.60 |
| | 0.001563 | 7.60 | 5 | 6.76 | 1 | 29.60 |
| | 0.000781 | 4.94 | 2.5 | 7.35 | 0.5 | 14.21 |
| | 0.000391 | 3.50 | 1.25 | 5.92 | 0.25 | 8.55 |
| | 0.000195 | 4.67 | 0.625 | 6.69 | 0.125 | 6.73 |
| | IC50 (μg/mL) = 0.0081 | | IC50 (μg/mL) = 59.74 | | IC50 (μg/mL) = 2.503 | |
| U-87MG | 0.025 | 45.58 | 640 | 98.03 | 16 | 76.01 |
| | 0.0125 | 19.31 | 320 | 98.43 | 8 | 85.10 |
| | 0.00625 | 18.61 | 160 | 82.81 | 4 | 63.60 |
| | 0.003125 | 19.10 | 80 | 2.45 | 2 | 43.00 |
| | 0.001563 | 15.53 | 40 | 15.15 | 1 | 20.84 |
| | 0.000781 | 17.11 | 20 | 15.72 | 0.5 | 2.72 |
| | 0.000391 | 14.75 | 10 | 5.82 | 0.25 | 0.10 |
| | 0.000195 | 11.52 | 5 | 2.94 | 0.125 | 1.91 |
| | 9.77 × 10⁻⁵ | 10.88 | 2.5 | 2.29 | 0.0625 | 2.27 |

| | | |
|---|---|---|
| IC50 (μg/mL) = 0.0262 | IC50 (μg/mL) = 76.85 | IC50 (μg/mL) = 2.751 |

TABLE 11
Inhibitory effects of Tea Product 1, EGCG monomer
(having a purity of 95%), and Epirubicin on
proliferation of human mammary gland/duct cancer
cell line BT-474 and human prostate cancer cell
line PC-3

| | Test tea samples prepared | | | | | |
|---|---|---|---|---|---|---|
| | from Tea Product 1 | | EGCG monomer test samples | | Epirubicin test samples | |
| | | Percent | | Percent | | Percent |
| | | growth | | growth | | growth |
| Cell | Concentrations | inhibition | Concentrations | inhibition | Concentrations | inhibition |
| line | (μg/mL) | (%) | (μg/mL) | (%) | (μg/mL) | (%) |
| BT-474 | 0.025 | 87.96 | 640 | 97.03 | 4 | 72.61 |
| | 0.0125 | 30.13 | 320 | 97.46 | 2 | 53.54 |
| | 0.00625 | −9.98 | 160 | 96.45 | 1 | 33.36 |
| | 0.003125 | 3.25 | 80 | 57.40 | 0.5 | 22.36 |
| | 0.001563 | 5.02 | 40 | −20.61 | 0.25 | 17.41 |
| | 0.000781 | 1.56 | 20 | −6.45 | 0.125 | 6.31 |
| | 0.000391 | 0.76 | 10 | −2.75 | 0.0625 | 2.50 |
| | 0.000195 | 8.48 | 5 | 2.62 | 0.03125 | 1.20 |
| | 9.77 × 10⁻⁵ | 4.57 | 2.5 | 6.51 | 0.015625 | 8.91 |
| | IC50 (μg/mL) = 0.0154 | | IC50 (μg/mL) = 74.75 | | IC50 (μg/mL) = 1.704 | |
| PC-3 | 0.025 | 89.38 | 640 | 92.53 | 32 | 85.63 |
| | 0.0125 | 38.30 | 320 | 96.31 | 16 | 81.64 |
| | 0.00625 | 7.29 | 160 | 86.71 | 8 | 82.75 |
| | 0.003125 | 16.75 | 80 | 74.43 | 4 | 82.00 |
| | 0.001563 | 21.98 | 40 | 40.11 | 2 | 74.03 |
| | 0.000781 | 18.05 | 20 | 26.03 | 1 | 66.13 |
| | 0.000391 | 16.04 | 10 | 19.64 | 0.5 | 55.10 |
| | 0.000195 | 12.28 | 7 | 19.10 | 0.25 | 47.99 |
| | 9.77 × 10⁻⁵ | 8.73 | 2.5 | 16.69 | 0.125 | 32.72 |

| | | |
|---|---|---|
| IC50 (μg/mL) = 0.0141 | IC50 (μg/mL) = 39.51 | IC50 (μg/mL) = 0.3336 |

TABLE 12
Inhibitory effects of Tea Product 1, EGCG monomer
(having a purity of 95%), and Etoposide on
proliferation of human leukemia cell line HL-60

| Test tea samples prepared | | EGCG monomer | | | |
|---|---|---|---|---|---|
| from Tea Product 1 | | test samples | | Etoposide test samples | |
| | Percent | | Percent | | Percent |
| | growth | | growth | | growth |
| Concentrations | inhibition | Concentrations | inhibition | Concentrations | inhibition |
| (μg/mL) | (%) | (μg/mL) | (%) | (μg/mL) | (%) |
| 0.025 | 73.96 | 640 | 91.13 | 32 | 100.46 |
| 0.0125 | 87.21 | 320 | 83.90 | 16 | 100.67 |
| 0.00625 | 64.68 | 160 | 73.03 | 8 | 100.08 |
| 0.003125 | 40.12 | 80 | 76.96 | 4 | 100.17 |
| 0.001563 | 17.29 | 40 | 44.21 | 2 | 100.27 |
| 0.000781 | 24.49 | 20 | 27.14 | 1 | 97.83 |
| 0.000391 | 5.58 | 10 | 16.22 | 0.5 | 82.91 |
| 0.000195 | −9.83 | 5 | 12.43 | 0.25 | 73.60 |
| 9.77 × 10⁻⁵ | −22.62 | 2.5 | 5.10 | 0.125 | 62.36 |

| | | |
|---|---|---|
| IC50 (μg/mL) = 0.0034 | IC50 (μg/mL) = 44.96 | IC50 (μg/mL) = 0.0855 |

TABLE 13
Inhibitory effects of Tea Product 1 and Glivec on
proliferation of human leukemia cell line K562

| Test tea samples | | | |
|---|---|---|---|
| prepared from Tea | | | |
| Product 1 | | Glivec test samples | |
| | Percent growth | | Percent growth |
| Concentrations | inhibition | Concentrations | inhibition |
| (μg/mL) | (%) | (μg/mL) | (%) |
| 0.01 | 88.53 | 0.5897 | 88.86 |
| 0.005 | 86.31 | 0.2949 | 81.53 |
| 0.0025 | 87.76 | 0.1474 | 66.22 |
| 0.00125 | 92.48 | 0.0737 | 55.29 |
| 0.000625 | 84.51 | 0.0369 | 35.73 |
| 0.000417 | 82.78 | 0.0184 | 17.68 |
| 0.000313 | 72.68 | | |
| 0.000139 | 86.59 | | |
| 4.63 × 10⁻⁵ | 88.59 | | |
| 1.54 × 10⁻⁵ | 23.79 | | |

| | |
|---|---|
| IC50 (μg/mL) = 0.00024 | IC50 (μg/mL) = 0.0655 |

TABLE 14
Inhibitory effects of Tea Product 1 and
Gefitinib on proliferation of human pancreatic
cancer cell line PANC-1

| Test tea samples | | | |
|---|---|---|---|
| prepared from | | | |
| Tea Product 1 | | Gefitinib test samples | |
| | Percent growth | | Percent growth |
| Concentrations | inhibition | Concentrations | inhibition |
| (μg/mL) | (%) | (μg/mL) | (%) |
| 0.01 | 77.48 | 14.3008 | 63.15 |
| 0.005 | 75.19 | 7.1504 | 41.19 |
| 0.0025 | 78.64 | 3.5752 | 33.11 |
| 0.00125 | 84.20 | 1.7876 | 28.42 |
| 0.000625 | 61.74 | 0.8938 | 20.09 |
| 0.000417 | 57.57 | 0.4469 | 20.42 |
| 0.000139 | 43.82 | | |
| 4.63 × 10⁻⁵ | 26.83 | | |
| 1.54 × 10⁻⁵ | −6.25 | | |

| | |
|---|---|
| IC50 (μg/mL) = 0.00026 | IC50 (μg/mL) = 3.7776 |

The data shown in Tables 7 to 14 indicate that Tea Product 1 has evident inhibitory effects against the proliferation of human ovarian cancer cell line SK-OV-3, human cervical cancer cell line Hela, human lung cancer cell line A549, human glioblastoma cell line U-87MG, human breast ductal carcinoma cell line BT-474, human prostate cancer cell line PC-3, human leukemia cell line HL-60, human gastric cancer cell line SMMC-7721, human colon cancer cell line HCT-116, human squamous cell carcinoma cell line A431, human leukemia cell line K562, human pancreatic cancer cell line PANC-1, and human gastric cancer cell line BGC-823, and such effects are generally concentration-dependent. Moreover, the inhibitory effects of Tea Product 1 are superior to those of EGCG monomer, and are matchable with those of the commercial chemotherapy drugs Cisplatin, Epirubicin, Etoposide, 5-fluorouracil, Gefitinib, and Glivec.

In view of the foregoing, the applicant inferred that the tea product of the present disclosure, which has satisfactory in vitro antitumor/anticancer activity against various tumor/cancer cells, may be further used in the treatment of cancer/tumor in animals.

### Example 4. In Vive Testing for Antitumor/Anticancer Activity of Tea Product of Present Disclosure

This example was conducted to investigate the potential in vivo antitumor/anticancer activity of the tea product of the present disclosure.

### A. Establishment of Animal Models

Two types of animal models (i.e. Animal Model 1 and Animal Model 2) were established.

The establishment of Animal Model 1 is addressed as follows. Male BALB/c nude mice (6 to 8 weeks of age, a body weight of 18 to 22 g, certificate number: SCXK (Hu) 2012-0002) were purchased from Shanghai Silaike Experiment Animal Co., Ltd and were acclimatized for one week. HepG2 cells (human liver cancer cells) mentioned in section C of Example 3 were cultivated in the corresponding complete medium (see Table 3) supplemented with 100 μg/mL penicillin and 100 μg/mL streptomycin in an incubator (37° C., 100% relative humidity, 5% CO₂). When adherent cancer/tumor cells reached 97% confluence, the medium was removed. Cell detachment was conducted using trypsin, and the detached cells were transferred to a centrifuge tube. Centrifugation was conducted, and the resulting supernatant was removed. Staining was performed with a 0.4% trypan blue, and it was determined that the cell viability was higher than 97%. A cell suspension having a concentration of 1l10⁷ cells/mL was prepared using PBS. 0.2 mL of the cell suspension was subcutaneously injected into each of the nude mice at a position posterior to the axilla. A mouse of Animal Model 1 was successfully established if a transplanted tumor having a size of 1 cm³ grew on the skin surface of such mouse 14 days after the inoculation.

The establishment of Animal Model 2 is described below. Male BALB/c nude mice (5 to 6 weeks of age, a body weight of 15 to 16 g, certificate number: SCXK (Hu) 2012-0002) were purchased from Shanghai Silaike Experiment Animal Co., Ltd and were acclimatized for one week. A cell suspension of HepG2 cells having a concentration of 1×10⁷ cells/mL was prepared using the same method as that for preparing the cell suspension for Animal Model 1. 0.2 mL of the cell suspension was subcutaneously injected into the back of each of 5 nude mice. When the resulting tumor grew to 1 cm³, a tumor tissue was collected under sterile conditions. The tumor tissue was cut into smaller pieces of tumor tissues (each of which had a size of 2 mm×2 mm×2 mm), followed by subcutaneous inoculation into the back of 10 nude mice other than the 5 nude mice treated with the cell suspension. When the resulting tumor grew to 1 cm³, the steps above were repeated to inoculate smaller pieces of tumor tissues into nude mice other than the 10 nude mice receiving subcutaneous inoculation, which were to serve as Animal Model 2. A mouse of Animal Model 2 was successfully established if a transplanted tumor having a size of 1 cm³ grew on the skin surface of such mouse.

### B. In Vive Antitumor Testing

Tea Product 1 and Conventional Tea Product obtained in section A of Example 3, as well as 5-fluorouracil mentioned in section B of Example 3, were examined for their in vivo antitumor activity.

The following three in vivo experiments were performed.

In the first experiment, nude mice of Animal Model 1 were divided into three groups, i.e. a control group (n=10), a 5-fluorouracil group (n=10), and a Tea Product 1 group (n=10). These three groups were respectively treated with distilled water, 5-fluorouracil, and Tea Product 1 at dosages shown in Table 15 for 35 days in a row.

In the second experiment, nude mice of Animal Model 2 were divided into three groups, i.e. a control group (n=12), a 5-fluorouracil group (n=12), and a Tea Product 1 group (n=12). These three groups were respectively treated with distilled water, 5-fluorouracil, and Tea Product 1 at dosages shown in Table 16 for 35 days in a row.

In the third experiment, nude mice of Animal Model 2 were divided into three groups, i.e. a control group (n=11), a Tea Product 1 group (n=11), and a Conventional Tea Product group (n=8). These three groups were respectively treated with distilled water, 5-fluorouracil, and Tea Product 1 at dosages shown in Table 17 for 35 days in a row.

In all the three experiments above, each of the nude mice was sacrificed 24 hours after the termination of the treatment. The tumor was collected and weighed. The antitumor effect of a respective test substance was evaluated by virtue of the percent tumor growth inhibition (%), which was calculated using the following Equation II:

E=[(F−G)/(F)]×100% (I)
E=percent tumor growth inhibition (%)
F=tumor weight of control group
G=tumor weight of respective test substance group

The experimental data are expressed as mean±SEM (standard error of the mean). The experimental data were subjected to one-way analysis of variance (ANOVA) using SPSS (Statistical Package for Social Sciences) software package version 11.5, such that the difference between the groups could be evaluated. The data regarding the tumor weights of the groups were subjected to t-test. Statistical significance is indicated by p<0.05.

### Results:

The results of the first, second, and third experiments are respectively shown in Tables 15 to 17 below.

First of all, the results of first experiment are addressed below.

TABLE 15
Results of first experiment

| | | | | | | Percent |
|---|---|---|---|---|---|---|
| | Number | | | | | tumor |
| | of | Dosage and | Body weight (g) | | Tumor | growth |
| | nude | administration | Before | After | weight | inhibition |
| Group | mice | route | treatment | treatment | (g) | (%) |
| Control | 10 | 0.6 mL of | 23.75 ± | 29.25 ± | 0.43 ± | / |
| | | distilled water | 1.03aA | 1.05aA | 0.22aA | |
| | | per day via | | | | |
| | | intragastric | | | | |
| | | gavage | | | | |
| 5-fluorouracil | 10 | 15.0 mg of | 23.65 ± | 72.90 ± | 0.08 ± | 80.84 |
| | | 5-fluorouracil/ | 0.45aA | 1.25cC | 0.06bB | |
| | | kg of body | | | | |
| | | weight per day | | | | |
| | | via | | | | |
| | | intra-abdominal | | | | |
| | | injection | | | | |
| Tea Product 1 | 10 | 0.6 mL of 10% Tea | 23.83 ± | 25.08 ± | 0.069 ± | 84.11 |
| | | Product 1 per | 0.56aA | 1.49bB | 0.02bB | |
| | | day via | | | | |
| | | intragastric | | | | |
| | | gavage | | | | |

When 5-fluorouracil Group and Tea Product 1 Group are compared with the control group, a different lower-case letter indicates p < 0.05, and a different upper-case letter indicates p < 0.01.
When Tea Product 1 Group is compared with 5-fluorouracil Group, a different lower-case letter indicates p < 0.05, and a different upper-case letter indicates p < 0.01.

As shown in Table 15, there is no significant difference between the percent tumor growth inhibition of Tea Product 1 (84.11%) and that of the chemotherapy drug 5-fluorouracil (80.84%) in nude mice of Animal Model 1, thereby indicating that Tea Product 1 is matchable with 5-fluorouracil in terms of the in vivo antitumor effect.

Secondly, the results of the second experiment are discussed as follows.

TABLE 16
Results of second experiment

| | | | | | | Percent |
|---|---|---|---|---|---|---|
| | Number | | | | | tumor |
| | of | Dosage and | Body weight (g) | | Tumor | growth |
| | nude | administration | Before | After | weight | inhibition |
| Group | mice | route | treatment | treatment | (g) | (%) |
| Control | 12 | 0.6 mL of | 17.30 ± | 21.45 ± | 0.067 ± | / |
| | | distilled water | 1.26aA | 1.65bB | 0.026aA | |
| | | per day via | | | | |
| | | intragastirc | | | | |
| | | gavage | | | | |
| 5-fluorouracil | 12 | 15.0 mg of | 17.22 ± | 24.32 ± | 0.034 ± | 48.96 |
| | | 5-fluorouracil/ | 1.06aA | 1.88aA | 0.017hB | |
| | | kg of body | | | | |
| | | weight per day | | | | |
| | | via | | | | |
| | | intra-abdominal | | | | |
| | | injection | | | | |
| Tea Product 1 | 12 | 0.6 mL of 10% Tea | 17.30 ± | 20.82 ± | 0.021 ± | 68.36 |
| | | Product 1 per | 1.18aA | 1.82bB | 0.015cC | |
| | | day via | | | | |
| | | intragastric | | | | |
| | | gavage | | | | |

When 5-fluorouracil Group and Tea Product 1 Group are compared with the control group, a different lower-case letter indicates p < 0.05, and a different upper-case letter indicates p < 0.01.
When Tea Product 1 Group is compared with 5-fluorouracil Group, a different lower-case letter indicates p < 0.05, and a different upper-case letter indicates p < 0.01.

As shown in Table 16, the percent tumor growth inhibition of Tea Product 1 (68.36%) in nude mice of Animal Model 2 is higher than that of the chemotherapy drug 5-fluorouracil (48.96%), and the difference therebetween has a significance level of 1%. Thus, the experimental result reveals that Tea Product 1 may have a superior in vivo antitumor effect compared to 5-fluorouracil.

Lastly, the results of the third experiment are described below.

TABLE 17
Results of third experiment

| | | | | | | Percent |
|---|---|---|---|---|---|---|
| | Number | | | | | tumor |
| | of | Dosage and | Body weight (g) | | Tumor | growth |
| | nude | administration | Before | After | weight | inhibition |
| Group | mice | route | treatment | treatment | (g) | (%) |
| Control | 11 | 0.6 mL of | 16.67 ± | 24.83 ± | 0.057 ± | / |
| | | distilled water | 1.14aA | 1.22aA | 0.010aA | |
| | | per day via | | | | |
| | | intragastirc | | | | |
| | | gavage | | | | |
| Tea Product 1 | 11 | 0.6 mL of 10% Tea | 16.35 ± | 21.48 ± | 0.021 ± | 63.60 |
| | | Product 1 per | 1.68aA | 3.15bB | 0.010cC | |
| | | day via | | | | |
| | | intragastirc | | | | |
| | | gavage | | | | |
| Conventional | 8 | 0.6 mL of 10% | 16.07 ± | 24.60 ± | 0.047 ± | 18.14 |
| Tea Product | | Conventional | 1.50aA | 1.69aA | 0.018bB | |
| | | Tea Product per | | | | |
| | | day via | | | | |
| | | intragastirc | | | | |
| | | gavage | | | | |

When Tea Product 1 Group and Conventional Tea Product Group are compared with the control group, a different lower-case letter indicates p < 0.05, and a different upper-case letter indicates p < 0.01.
When Tea Product 1 Group is compared with Conventional Tea Product Group, a different lower-case letter indicates p < 0.05, and a different upper-case letter indicates p < 0.01.

As shown in Table 17, the percent tumor growth inhibition of Tea Product 1 (63.60%) in nude mice of Animal Model 2 is higher than that of Conventional Tea Product (18.14%), and the difference therebetween has a significance level of 1%.

In view of the foregoing, the applicant confirmed that the tea product of the present disclosure, which has satisfactory in vivo antitumor activity, can be used in the treatment of cancer/tumor in animals.

### Example 5. Further In Vivo Testing for Antitumor Activity of Tea Product of Present Disclosure

This example was performed to further demonstrate the in vivo antitumor activity of the tea product of the present disclosure.

### A. Preparation of Another Antitumor/Anticancer Tea Product of Present Disclosure

Tea leaves of Camellia sinensis Strain 2 obtained in Example 1 were used to prepare Tea Product 2 according to the method described in section 2 of “General Experimental Procedures”.

### B. In Vivo Antitumor Testing

Each of Conventional Tea Product (obtained in section A of Example 3) and Tea Product 2 (obtained in section A of this example) was subjected to the following process, so as to prepare a formulation for the testing. A respective one of Conventional Tea Product and Tea Product 2 was mixed with distilled water of 80° C. at a ratio of 3:100, followed by leaving standstill for 5 minutes. Ultrasonic extraction was conducted at 200 W for 10 minutes twice. The two filtrates were combined to obtain a respective formulation.

Clean-grade male Wistar rats (5 weeks of age, a body weight of 140±10 g) were purchased from Beijing Tong Lihua Experimental Animal Technical Co., Ltd. (SCXK2012-0001). After the rats were acclimatized for one week, the rats were randomly divided into four groups (n=10 for each), i.e. a positive control group, a control group, a Conventional Tea Product group, and a Tea Product 2 group. From 2^{nd} to 12^{th} weeks during the testing, diethylnitrosamine was intra-abdominally injected into each of the rats in the control group, the Conventional Tea Product group, and the Tea Product 2 group twice a week at a dose of 25 mg/kg of body weight. From 1^{st} to 20^{th} weeks during the testing, the rats in the control group, the Conventional Tea Product group, and the Tea Product 2 group were respectively treated with distilled water (ad libitum), Formulation of Conventional Tea Product (ad libitum), and Formulation of Tea Product 2 (ad libitum).

The rats in the positive control group received no diethylnitrosamine and no treatment of test substance.

At the end of the 20^{th} week during the testing, all the rats were sacrificed. The liver tumorigenesis of each rat was observed, and the number of tumors in each rat was counted. Furthermore, the size of each tumor (i.e. length, width, and height) was measured using a vernier caliper, such that the tumor volume (mm³) could be calculated.

The experimental data are expressed as mean±SEM. The experimental data were subjected to ANOVA using SPSS software package version 11.5, such that the difference between the groups could be evaluated. The data regarding the tumor number and the tumor volume of the groups were subjected to Fisher's exact test. Statistical significance is indicated by p<0.05.

### Results:

The results of the in vivo antitumor testing are shown in Table 18 below.

TABLE 18
Results of in vivo antitumor testing

| | Number | Number of rats | | Tumor number | |
|---|---|---|---|---|---|
| | of rats | undergoing | Tumor | per rat | |
| | for | liver | incidence | undergoing | Tumor volume |
| Group | testing | tumorigenesis | rate | tumorigenesis | (mm³) |
| Positive | 10 | 0 | 0 | 0 | 0 |
| control | | | | | |
| Control | 10 | 9 | 90% | 83.4 ± 61.9aA | 99.7 ± 29.4aA |
| Conventional | 10 | 10 | 100% | 36.2 ± 18.9cC | 19.7 ± 14.0bAB |
| Tea Product | | | | | |
| Tea Product 2 | 10 | 6 | 60% | 13.0 ± 15.5dD | 4.9 ± 5.9cC |

When Conventonal Tea Product Group and Tea Product 2 Group are compared with the control group, a different lower-case letter indicates p < 0.05, and a different upper-case letter indicates p < 0.01.
When Tea Product 2 Group is compared with Conventional Tea Product Group, a different lower-case letter indicates p < 0.05, and a different upper-case letter indicates p < 0.01.

As shown in Table 18, the tumor incidence rate of the Tea Product 2 group is only 60%. Compared to the tumor incidence rate of the control group (90%), that of the Tea Product 2 group is lower by 33.3%. Further regarding the Tea Product 2 group, the tumor number per rat undergoing tumorigenesis is only about 13, which is significantly lower by 84.41% (p<0.01) compared to that of the control group (about 84.3). Likewise, the tumor incidence rate and tumor number of the Tea Product 2 group are evidently lower than those of the Conventional Tea Product group. Therefore, it can be demonstrated that Tea Product 2 has a satisfactory tumor-treating effect.

In addition, the tumor volume of the Tea Product 2 group is only about 4.9 mm³ and is hence small. Compared to the tumor volume of the control group (99.7 mm³), that of the Tea Product 2 group is lower by 95.09% (p<0.05). Likewise, the tumor volume of the Tea Product 2 group is evidently lower than that of the Conventional Tea Product group. Thus, it can be revealed that Tea Product 2 has an excellent in vivo tumor-treating effect.

In view of the foregoing, it can be once demonstrated that the tea product of the present disclosure has in vivo antitumor/anticancer activity and hence may be used in treatment of animals. Accordingly, the selective breeding method of the present disclosure indeed can be employed to produce an antitumor/anticancer Camellia sinensis strain.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A selective breeding method of an antitumor/anticancer Camellia sinensis strain, comprising:
conducting a hybridization process so as to obtain progenies of Camellia sinensis;
subjecting the progenies of Camellia sinensis to determination of bioactive ingredient contents, wherein the determination of bioactive ingredient contents is conducted using dry leaves of the progenies of Camellia sinensis; and
selecting, from the progenies of Camellia sinensis, an antitumor/anticancer Camellia sinensis strain which meets the following five standards of bioactive ingredients: a total catechin content ≧22 wt %, an EGCG content ≧12 wt %, a catechin C content ≧2 wt %, an ECG content ≧3.5 wt %, and a caffeine content ≧4.5 wt % based on the total content of essential bioactive ingredients determined.

2. The selective breeding method as claimed in claim 1, wherein the hybridization process comprises:
subjecting at least one paternal generation of Camellia sinensis and a maternal generation of Camellia sinensis to hybridization, wherein the paternal and maternal generations of Camellia sinensis meet at least one of the five standards of bioactive ingredients, the number of the standards which the maternal generation of Camellia sinensis meets is higher than or equal to the number of the standards which the at least one paternal generation of Camellia sinensis meets, the standards which the paternal and maternal generations of Camellia sinensis meet are in a complementary relationship.

3. The selective breeding method as claimed in claim 2, wherein the hybridization process further comprises:
obtaining seedlings from offsprings of Camellia sinensis produced by the hybridization of the paternal and maternal generations of Camellia sinensis and sowing the seedlings after ripening of fruits of the offsprings of Camellia sinensis; and
subjecting the seedlings to transplantation after sowing.

4. The selective breeding method as claimed in claim 2, wherein the paternal and maternal generations of Camellia sinensis are cultivated in a field that has a relative humidity of higher than or equal to 90%.

5. The selective breeding method as claimed in claim 4, wherein the field is at an elevation of lower than or equal to 300 meters above sea level, and receives, compared to normal fields, 550-650 Kg more of potassium sulfate, 300-400 Kg more of calcium superphosphate, and 8500-9500 Kg more of fresh stems and leaves of Cassia obtusifolia L. per hectare each year.

6. The selective breeding method as claimed in claim 2, wherein the maternal generation of Camellia sinensis and at least one of the at least one paternal generation of Camellia sinensis are geographically distant strains which contain 25% or more of tea polyphenols on a dry matter basis of Camellia sinensis leaves.

7. The selective breeding method as claimed in claim 2, wherein at least one of the at least one paternal generation of Camellia sinensis is obtained by hybridizing geographically distant strains of Camellia sinensis which contain 25% or more of tea polyphenols on a dry matter basis of Camellia sinensis leaves.

8. The selective breeding method as claimed in claim 3, wherein terminal buds of the seedlings growing to a height ranging from 20 cm to 30 cm are removed after sowing and before transplantation.

9. The selective breeding method as claimed in claim 3, wherein each of the seedlings growing to have a height of equal to and higher than 35 cm is transplanted during a period of time spanning from November to subsequent February.

10. The selective breeding method as claimed in claim 3, wherein the progenies of Camellia sinensis obtained after cultivation of the seedlings for three years are subjected to the determination of bioactive ingredient contents for three consecutive years, and each of the progenies of Camellia sinensis which meets the five standards of bioactive ingredients for three consecutive years is the antitumor/anticancer Camellia sinensis strain.

11. The selective breeding method as claimed in claim 10, wherein each of the progenies of Camellia sinensis which meets the five standards of bioactive ingredients in the same harvest season for three consecutive years is the antitumor/anticancer Camellia sinensis strain.

12. An antitumor/anticancer tea product produced from the antitumor/anticancer Camellia sinensis strain obtained using the selective breeding method as claimed in claim 1.

13. The antitumor/anticancer tea product as claimed in claim 12, which is for treating a cancer/tumor selected from the group consisting of liver cancer, ovarian cancer, cervical cancer, lung cancer, glioblastoma, mammary gland cancer, prostate cancer, leukemia, gastric cancer, colon cancer, squamous cell carcinoma, pancreatic cancer, and combinations thereof.

14. A pharmaceutical composition comprising the antitumor/anticancer tea product as claimed in claim 12.

15. The pharmaceutical composition as claimed in claim 14, which is for treating a cancer/tumor selected from the group consisting of liver cancer, ovarian cancer, cervical cancer, lung cancer, glioblastoma, mammary gland cancer, prostate cancer, leukemia, gastric cancer, colon cancer, squamous cell carcinoma, pancreatic cancer, and combinations thereof.

16. A supplement comprising the antitumor/anticancer tea product as claimed in claim 12.
